(19) United States
(12) Patent Application Publication
(10) Pub. No.: US 2021/0332369 A1
(43) Pub. Date: Oct. 28, 2021

(54) USE OF CBX4 AS TARGET FOR ACTIVATION OF HIV-1 LATENT INFECTION

(71) Applicant: SUN YAT-SEN UNIVERSITY, Guangdong (CN)

(72) Inventors: Hui ZHANG, Guangdong (CN); Cancan CHEN, Guangdong (CN)

(73) Assignee: SUN YAT-SEN UNIVERSITY, Guangdong (CN)

(21) Appl. No.: 16/321,831

(22) PCT Filed: Aug. 29, 2017
(86) PCT No.: PCT/CN2017/099466

§ 371 Date: Jan. 30, 2019

(51) Int. Cl. C12N 15/113; A61P 31/18

(52) U.S. Cl. CPC C12N 15/1138; C12N 2310/531; C12N 2310/14; A61P 31/18

(57) ABSTRACT

The present invention relates to a use of CBX4 as a target for activation of HIV-1 latent infection. The present invention found by research that, inhibiting CBX4 causes a good ability for activation of HIV-1 latent, a knockdown of CBX4 can effectively promote a transcription of LTR of HIV-1; and afterwards the present invention has found that, by the knockdown of CBX4 in a J-lat 10.6 cell model of HIV-1 latent infection, GFP gene expression is up-regulated, and HIV-1 can be effectively activated. An overexpression of the CBX4 protein in TZM-bl cells can effectively decrease a transcriptional activity of LTR of HIV-1. It is found by further detection that, decreased expression of CBX4 protein will reduce a degree of enrichment of H3K9 trimethylation and H3K27 trimethylation of LTR of HIV-1, thereby activating HIV-1 latent infection.

### BACKGROUND

### Technical Field

The present invention relates to the technical field of disease-related functional targets, specifically, to the technical field of a target for activation of HIV-1 latent infection, and more specifically, to a use of CBX4 as a target for activation of HIV-1 latent infection.

### Description of Related Art

HIV-1 virus is a lentivirus that infects human immune system cells. It attacks human T lymphocytes, destroys the human immune system, and eventually leads to an emergence of an acquired immunodeficiency syndrome “AIDS”. This virus was first discovered in 1981, and for more than 30 years afterwards, researchers have been working on the control and clearance of this virus, hoping to cure this disease. The current conventional therapeutic method is cART, short for combination antiretroviral therapy, which can effectively inhibit replication of virus, thereby controlling a viral load in peripheral blood of a patient to a lower level. However, a current general treatment method can only control the virus to a certain extent and cannot effectively clean the virus. The reason is because HIV-1 may lurk in cells to form HIV-1 reservoirs. Conventional drugs can not affect these reservoirs, and once the patient discontinues treatment, they will be activated for various reasons, thereby leading to a relapse of illness. To sum up, an existence of HIV-1 reservoir has become a huge obstacle to the cure of HIV-1. How to effectively clean the HIV-1 reservoir is one of the research hotspots in the field of HIV-1. Today's mainstream method “shock and kill” is the idea of activating first and then killing. Therefore, developing a safe and efficient activator for HIV-1 latent infection is one of the current problems that have yet to be solved. A variety of activators for HIV-1 latent infection have been developed, and they can be classified into following types according to their targets, including: histone deacetylase inhibitor (HDACi), histone methyltransferase inhibitor (HMTi), DNA methyltransferase inhibitor (DNMTi), protein kinase C activator (PKC), and bromodomain inhibitor.

CBX4 belongs to a CBX protein family and is an important component of the polycomb repressive complex 1 (PRC1). The CBX protein family includes five members: CBX2, CBX4, CBX6, CBX7, and CBX8. Their N-terminus has a CHROMO domain, which has a binding ability of H3K27 trimethylation; while the C-terminus has a conserved region called C-Box, which is responsible for an assembly of the complex PRC1. In particular, CBX4 not only has a certain affinity for H3K27 trimethylation, but also exhibits a significant affinity with H3K9 trimethylation. A function of CBX4 reported in the prior art is mainly involved in regulation of individual development, stem cell differentiation and tumorigenesis. So far, no studies have revealed that CBX4 is a novel target for activation of HIV-1 latent infection.

### SUMMARY

An object of the present invention is to provide a use of CBX4 as a brand new target for activation of HIV-1 latent infection. In order to realize the above-described object, the present invention is realized by the following technical solution.

The present invention found by research that, inhibiting CBX4 causes a good ability for activation of HIV-1 latent. In the present invention, by a knockdown of an expression quantity of CBX4 gene in TZM-bl cells, detecting a transcriptional level of LTR of HIV-1 using dual-luciferase report gene, it is found that a knockdown of CBX4 can effectively promote a transcription of LTR of HIV-1. Afterwards the present invention has an effective knockdown of expression situation of CBX4 protein in a J-lat 10.6 cell model of HIV-1 latent infection, and it is found that J-lat 10.6 cells can be effectively activated and GFP gene expression is up-regulated. The present invention has also constructed a CBX4 expression plasmid, and the CBX4 protein is overexpressed in the TZM-bl cells. It is found by the dual-luciferase report gene assay that, as the expression of CBX4 protein increases, a transcriptional activity of LTR of HIV-1 decreases accordingly, and a relationship between the two has a concentration gradient effect. It is found by further detection that decreased expression of CBX4 protein will reduce a degree of enrichment of H3K9 trimethylation and H3K27 trimethylation of LTR of HIV-1, thereby activating HIV-1 latent infection. Therefore, the present invention claims a use of CBX4 as a target for activation of HIV-1 latent infection.

Since CBX4 may be used as the target for activation of HIV-1 latent infection, a compound or a derivative thereof which inhibits a CBX4 gene or a CBX family gene, or a compound or a derivative thereof which degrades a CBX4 protein or a CBX family protein can be prepared into an HIV-1 latent infection activator.

Said derivative is a pharmaceutically acceptable salt or ester of the compound.

Said pharmaceutically acceptable salt is a sodium salt, a potassium salt, a calcium salt, a ferric salt, a ferrous salt or a zinc salt.

Said pharmaceutically acceptable ester is an ethyl ester or a butyl ester.

Compared with the prior art, the present invention has following beneficial effect:

the present invention has first found that CBX4 can be used as a target for activation of HIV-1 latent infection, providing a strong theoretical basis and practical basis for further development of HIV-1 related drugs.

### BRIEF DESCRIPTION OF THE DRAWINGS

FIG. 1 shows an efficient knockdown of an expression of a CBX4 gene in TZM-bl cells, which can effectively promote a transcriptional activity of LTR of HIV-1, wherein chart A shows a knockdown efficiency of si-CBX4 in this cell line which is detected by q-PCR; and chart B shows detection and analysis of LTR transcriptional activity detected by dual-luciferase report gene.

FIG. 2 shows that a knockdown of a CBX4 gene in J-lat 10.6 HIV-1 latent infection cells by using shRNA-CBX4 can effectively activate a HIV-1 latent infection reservoir, wherein chart A shows an efficiency of the knockdown of CBX4 by shRNA-CBX4 in a J-lat 10.6 cell line which is detected by q-PCR; and chart B shows an activation effect of HIV-1 latent infection in this cell model after detecting the knockdown of CBX4 by utilizing flow cytometry.

FIG. 3 shows that a CBX4 expression plasmid is constructed, and that an overexpression of the CBX4 protein in TZM-bl cells can effectively decrease a transcription of LTR of HIV-1, wherein chart A shows an expression effect of a constructed overexpression plasmid pcDNA3.1-CBX4-FLAG, detected by Western Blot technique; and chart B shows a gradient effect between LTR transcriptional activity and CBX4 overexpression, by using dual-luciferase report gene assay.

FIG. 4 shows that a decreased expression of CBX4 protein will reduce a degree of enrichment of H3K9 trimethylation and H3K27 trimethylation of LTR of HIV-1, thereby activating HIV-1 latent infection.

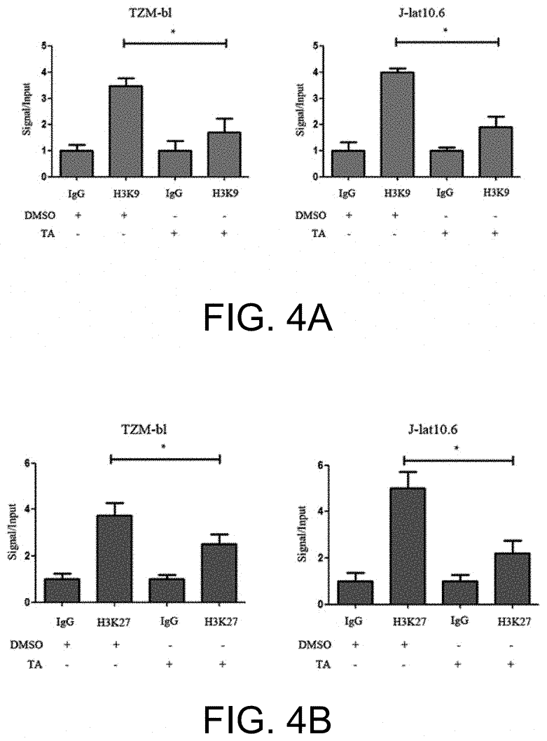

### DESCRIPTION OF THE EMBODIMENTS

The present invention is further described in detail below in combination of accompanying drawings and specific embodiments of the specification, and the embodiments are only used to explain the present invention, but not used to limit the scope of the present invention. The test methods used in the following embodiments, unless otherwise specified, are all conventional methods; and the materials, reagents, and etc. used, unless otherwise specified, are commercially available reagents and materials.

### Embodiment 1

A knockdown of a CBX4 gene expression can effectively promote a transcriptional activity of LTR of HIV-1, and a specific experimental method is as follows:

(1) well-grown TZM-bl cells were taken and inoculated in a 24 well-plate; and a medium used was a complete medium: high glucose DMEM, 10% fetal bovine serum and 1% double antibody, with a culture condition of 5% carbon dioxide, at 37° C.;

(2) after 24 hours of adherence, a pcDNA3.1-Tat plasmid was co-transfected with siRNA-CBX4 or siRNA-NC respectively, and a culture was continued under the condition in (1);

(3) after 48 hours, a cell pellet was collected, and a part of it was taken to extract RNA was by a Trizol method, and cDNA was reversed and obtained; a CBX4-specific Q-PCR primer was used to detect an expression level of mRNA of CBX4, and a knockdown efficiency of siRNA-CBX4 was determined;

(4) the remaining cells were taken, and after a lysis, a method of dual fluorescein plum reporter gene assay was used to detect a transcriptional activity of LTR of HIV-1 in different transfection treatment groups; and

(5) the transcriptional activity of LTR of HIV-1 after the knockdown of CBX4 was compared.

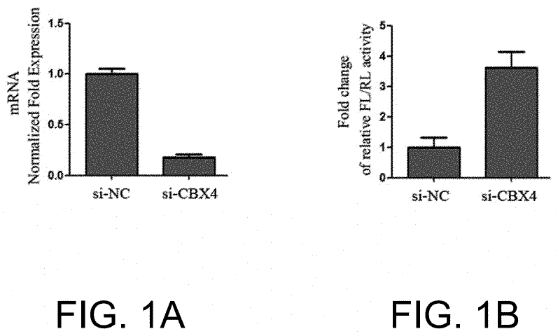

Experimental results are shown in FIG. 1. As can be seen from FIG. 1, in the TZM-bl cells, siRNA-CBX4 can have an effective knockdown of the endogenous CBX4 gene, and its knockdown efficiency is about 80%. It is found by comparison that the knockdown of CBX4 can effectively improve the transcriptional level of LTR of HIV-1.

### Embodiment 2

Inhibition of CBX4 in J-lat 10.6 HIV-1 latent infection cells effectively activates a HIV-1 latent infection reservoir. A specific experimental method is as follows:

(1) a specific base sequence was synthesized, shRNA and PLKO.1-CBX4 against CBX4 were constructed by annealing, and a sequencing was performed to confirm that the sequence was correct;

(2) well-grown human kidney cell line 239T cells were taken and inoculated in a 10 cm flat bottom plate; and a medium used was a complete medium: high glucose DMEM, 10% fetal bovine serum and 1% double antibody, with a culture condition of 5% carbon dioxide, at 37° C.;

(3) after 24 hours of adherence, PLKO.1-CBX4 or PLKO.1-NC and psPAX2, VSV-G were transfected into the cells, and a culture was continued under the condition in (2);

(4) after 48 hours, a supernatant was collected, virus particles were concentrated by centrifugation using PEG-6000, and a virus P24 was detected using an ELISA P24 kit;

(5) well-grown J-lat 10.6 cells were taken, and after being counted, were infected with the virus obtained in (4), a dosage of the virus was about 2-3×106/ml cells infected by virus in each 10 cm plate; (a pro-infective agent polybrene was used during infection);

(6) after 6 hours of infection, a solution was changed, washing was performed for three times with PBS, a supernatant was discarded, and a culture was performed with a 1640 medium (10% fetal bovine serum, 1% double antibody), with a culture condition of 5% carbon dioxide, at 37° C.;

(7) on the next day, an appropriate amount of puromycin was added into the medium at a concentration of about 1-2 μg/ml, 5% carbon dioxide, and was incubated at 37° C. for 2 weeks; the 1640 medium containing puromycin was regularly changed to obtain J-lat 10.6 cells stably infected with shRNA-CBX4;

(8) a part of the cells was taken to RNA using Trizol lysis, cDNA was reversed and obtained, and a CBX4-specific Q-PCR primer was used to detect an expression level of mRNA of CBX4; and

(9) an expression level of GFP of corresponding cells was detected by a flow cytometer, and a statistical chart was graphed for result analysis.

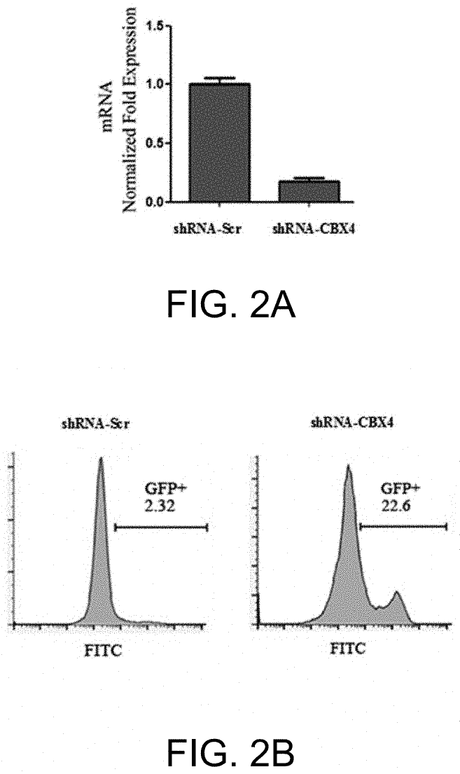

Experimental results are shown in FIG. 2. It can be seen from FIG. 2 that an effective knockdown of CBX4 in the J-lat 10.6 HIV-1 latent infection cells can effectively activate a HIV-1 latent infection reservoir.

### Embodiment 3

Overexpression of CBX4 protein in TZM-bl cells can inhibit a transcriptional activity of LTR of HIV-1. A specific experimental method is as follows:

(1) a CBX4 cDNA plasmid template was ordered, an appropriate primer was designed, and a CBX4 overexpression plasmid, pcDNA3.1-CBX4-FLAG, was constructed;

(2) well-grown TZM-bl cells were taken and paved in a 6-well transparent plate with 1×106 cells per well; a medium used was a complete medium: high glucose DMEM, 10% fetal bovine serum and 1% double antibody, with a culture condition of 5% carbon dioxide, at 37° C.;

(3) after 24 hours of adherence, a plasmid pcDNA3.1-CBX4-FLAG was transfected, and a culture was continued under the condition in (2);

(4) after 48 hours, the cells were collected and were lysed with a RIPA lysate, a CBX4 overexpression level was detected by western blot, to determine an effect of the overexpression plasmid pcDNA3.1-CBX4-FLAG;

(5) the well-grown TZM-bl cells were taken and paved in a 24-well transparent plate with 1×105 cells per well; the medium used was a complete medium: high glucose DMEM, 10% fetal bovine serum and 1% double antibody, with a culture condition of 5% carbon dioxide, at 37° C.;

(6) after 24 hours of adherence, different concentrations of pcDNA3.1-CBX4-FLAG plasmid and pcDNA3.1-Tat plasmid were transfected respectively, and a pcDNA3.1-CBX4-FLAG gradient was set from 0 to 500 ng, a consistency of the plasmids was adjusted with pcDNA3.1, and a culture was continued under the condition in (2); and

(7) after 48 hours, the cells were collected and were lysed with the lysate, a transcriptional activity of LTR of HIV-1 in different transfection treatment groups was detected by a method of dual fluorescein plum reporter gene assay.

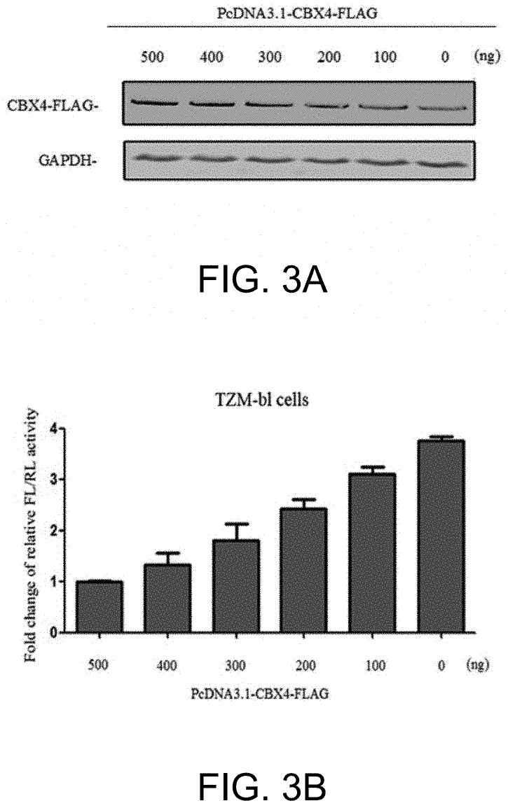

Experimental results are shown in FIG. 3. As can be seen from FIG. 3, as an expression quantity of CBX4 protein increases, the transcriptional activity of LTR of HIV-1 gradually decreases.

### Embodiment 4

Decreased expression of CBX4 protein will reduce a degree of enrichment of H3K9 trimethylation and H3K27 trimethylation of LTR of HIV-1. A specific experimental method is as follows:

(1) well-grown TZM-bl cells were taken and inoculated in a 10 cm flat bottom plate; a medium used was a complete medium: high glucose DMEM, 10% fetal bovine serum and 1% double antibody, with a culture condition of 5% carbon dioxide, at 37° C.;

(2) after 24 hours of adherence, a pcDNA3.1-Tat plasmid was co-transfected with siRNA-CBX4 or siRNA-NC respectively, and a culture was continued under the condition in (1);

(3) after 48 hours, a cell pellet was collected, and a degree of enrichment of H3K9 trimethylation and H3K27 trimethylation of LTR of HIV-1 was detected by ChIP using H3K9 trimethylation and H3K27 trimethylation ChIP antibody;

(4) well-grown J-lat 10.6 cells were taken, and after being counted, were infected with shRNA-SCR and shRNA-CBX4 respectively, a dosage of virus was about 2-3×10⁶/ml cells infected by virus in each 10 cm plate; (a pro-infective agent polybrene was used during infection);

(5) after 6 hours of infection, a solution was changed, washing was performed for three times with PBS, a supernatant was discarded, and a culture was performed with a 1640 medium (10% fetal bovine serum, 1% double antibody), with a culture condition of 5% carbon dioxide, at 37° C.;

(6) on the next day, an appropriate amount of puromycin was added into the medium at a concentration of about 1-2 μg/ml, 5% carbon dioxide, and was incubated at 37° C. for 2 weeks; the 1640 medium containing puromycin was regularly changed to obtain J-lat 10.6 cells stably infected with shRNA-CBX4;

(7) a cell pellet was collected, and a degree of enrichment of H3K9 trimethylation and H3K27 trimethylation of LTR of HIV-1 was detected by ChIP using H3K9 trimethylation and H3K27 trimethylation ChIP antibody; and

(8) the degree of enrichment of H3K9 trimethylation and H3K27 trimethylation of LTR of HIV-1 before and after the knockdown of CBX4 was compared and analyzed.

CBX4 is an effective target for activation of HIV-1 latent infection, a compound and a derivative thereof, which inhibit the CBX4 (and CBX family) gene or degrade the CBX4 (and CBX family) protein, particularly a pharmaceutically acceptable salt or ester, such as sodium salt, potassium salt, calcium salt, ferric salt, ferrous salt, zinc salt, ethyl ester, butyl ester, etc., can also activate HIV-1 latent infection, which is convenient for the follow-up treatment of HIV.

1. A use of CBX4 as a target for activation of HIV-1 latent infection.

2. The use according to claim 1, wherein the use is specifically of preparing a compound or a derivative thereof which inhibits a CBX4 gene or a CBX family gene, or a compound or a derivative thereof which degrades a CBX4 protein or a CBX family protein into an HIV-1 latent infection activator.

3. The use according to claim 2, wherein the derivative is a pharmaceutically acceptable salt or ester of the compound.

4. The use according to claim 3, wherein the pharmaceutically acceptable salt is a sodium salt, a potassium salt, a calcium salt, a ferric salt, a ferrous salt or a zinc salt.

5. The use according to claim 3, wherein the pharmaceutically acceptable ester is an ethyl ester or a butyl ester.
